(19) United States
(12) Patent Application Publication
(10) Pub. No.: US 2007/0162040 A1
(43) Pub. Date: Jul. 12, 2007

(54) Spinal distraction and endplate preparation device and method

(75) Inventors: John Grabowski, Bloomington, MN (US); Kevin V. Guenther, Carver, MN (US)

Correspondence Address: WOOD, HERRON & EVANS (ZIMMER SPINE), 2700 CAREW TOWER, 441 VINE STREET, CINCINNATI, OH, 45202, US

(73) Assignee: Zimmer Spine, Inc., Minneapolis, MN (US)

(21) Appl. No.: 11/294,749

(22) Filed: Dec. 6, 2005

(51) Int. Cl. A61B 17/58

(52) U.S. Cl. 606/90

(57) ABSTRACT

A surgical instrument system for distraction and endplate preparation of two adjacent vertebrae during a spinal stabilization procedure includes a distraction device with two arms each having two spaced-apart vertebra engaging portions at a distal end and capable of bilaterally engaging the vertebra. The system further includes a template having a guide portion and one or more spacer portions for inserting into the disc space between the two vertebrae and maintaining a predetermined distance between the vertebrae. The system further includes an endplate preparation device adapted to modify the adjacent endplates of the first and second vertebrae for receiving a spinal stabilization implant, the endplate preparation device having a range of motion that may be limited by the guide in at least one dimension.

### FIELD OF THE INVENTION

The present invention relates generally to surgical procedures for spinal stabilization and more specifically to devices and methods for preparing the intervertebral disc space between adjacent vertebra for receiving a spinal implant. More particularly, the present invention is especially suited for disc space preparation and implant insertion into a disc space from an anterior surgical approach to the spine.

### BACKGROUND OF THE INVENTION

The structures of the spine include vertebral bodies, vertebral discs, ancillary ligaments, and facet joints. The vertebral discs are cushion-like separators between the vertebrae that permit movement of the spine. Each normal human vertebral disc is made up of an outer circumferential ring of laminated fibers made of an elastic material. This ring is known as the annulus and has a thickness ranging between 5-15 mm. The annulus surrounds a nucleus center of the vertebral disc.

If the material located at the center of the nucleus is diminished by age, damage, or disease, the fibers of the annulus become lax and the vertebral disc may bulge abnormally. With extreme bulging, torsional instability in the vertebral discs can develop and a de-lamination of the layers of annulus fibers can result. In many patients, this cascade of disc degeneration results in segmental pain. On the other hand, if the nucleus remains well hydrated and only patches of the annulus fiber layers become weakened or torn by an accident, a loose radiating channel may develop through adjacent patches and provide an escape route for portions of the high-pressure material of the nucleus. This escape of tissue and byproducts outside the annulus is generally known as a herniated disc.

The escape or leaking of byproducts produced by the nucleus through an annulus defect may reach nerve endings found in the outer layers of the vertebral disc and cause severe back pain. Vertebral fusion is a commonly used surgical techniques for successfully treating this type of spinal problem.

Vertebral fusion alleviates back pain primarily by stopping all relative motion of the involved spinal segments. Vertebral fusion operations are regularly performed and a significant clinical improvement is observed in most cases. As such, the need for improved, safe, effective, simpler and less invasive fusion techniques and devices continues to grow. One method of fusion is to insert a bone, bone substitute, prosthesis, or a device containing bone into a surgically prepared vertebral disc space. Preparing for the bone or device insertion requires that the disc space be forced open and maintained open while the vertebral disc nucleus is removed. Several types of vertebral disc space distraction or spreading devices have been developed for this purpose.

While the disc space is maintained open by a distraction instrument, the surgeon works deeply within the space to remove dead or herniated tissue or bone spurs. As such, it is desirable to be able to maintain a desired distraction. After the herniated tissue is removed, the surgeon prepares the endplates of the vertebra for receiving an implant. This is usually done by breaking through, or cutting into, the hardened endplate surfaces of vertebral bone so as to allow an interposed bone graft or implant to come into direct contact with vascularized cancellous bone tissue. This enables blood flow through material placed in the intervertebral space, which in turn initiates the growth of new bone across the intervertebral space. This process allows for the incorporation of inserted bone grafts or implants into the two respective adjacent vertebral surfaces so that they become one continuous and rigid segment of bone over time.

Such continuous distraction can be accomplished by several techniques and apparatuses. The prosthesis or bone insert to be implanted can itself be wedge shaped and driven into the vertebral disc space which creates its own distraction of the vertebral bodies. However, the potential for expulsion of the inserted prostheses or bone insert exists. Also, the force needed to seat the insert into the vertebral disc space can be excessive. Further, the deep dissection of the vertebral disc space has to be performed before driving the insert into its final position. Because distraction is needed while the dissection takes place, a separate distraction device may be needed.

The most common instruments used to apply a distracting force between adjacent vertebral bodies attach directly to the vertebral bodies and neural arches or are placed inside the disc space off to a side between adjacent endplates of the vertebral bodies. A typical device used to spread the neural arches and the associated vertebral disc space of adjacent vertebrae is a lamina spreader. Such a device has opposing members that hook into the laminas that lie above and below the disc space. These hooks are forced apart by an attached rack and pinion mechanism or by a hinged appliance having a ratchet lock. Similarly, intradiscal spreaders apply force directly via blade members to the endplates of the vertebrae in order to spread them apart. Since the distraction portions must be unobtrusive to the surgeon, they must be small and placed laterally out of the way.

All of these distraction devices present obstructions to the open surgical field. These devices also make it difficult to precisely maintain a desired distraction during preparation of the vertebral endplates and subsequent placement of an implant. The invention disclosed herein is aimed at providing an improved distraction device.

### SUMMARY OF THE INVENTION

Generally, the invention provides a surgical instrument system for distraction and endplate preparation of adjacent vertebrae during a spinal stabilization procedure. In one embodiment, the surgical instrument system includes a first distraction arm with a first pair of spaced apart vertebra engaging portions at a distal end of the first arm and adapted to bilaterally engage a first vertebra. The system further includes a second distraction arm that includes a second pair of spaced apart vertebra engaging portions at a distal end of the second arm and adapted to bilaterally engage a second vertebra adjacent the first vertebra. The distal ends of the first and second arms are movable with respect to one another to displace the vertebra away from each other. The system further includes a template sized to be inserted between the first and second vertebrae and between each pair of the spaced apart vertebra engaging portions and adapted to bilaterally engage both the first and second vertebrae and maintain the vertebrae apart at a predetermined distance from each other.

In another embodiment, the surgical system includes a distraction device and an endplate preparation device. The distraction device includes a first distraction arm having first and second spaced apart vertebra engaging portions at a distal end capable of bilaterally engaging a first vertebra. The distraction device also includes a second distraction arm having first and second spaced apart vertebra engaging portions at a distal end capable of bilaterally engaging a vertebra. The second distraction arm may also be movable with respect to the first distraction arm for separating adjacent endplates of the first and second vertebrae. The distraction device also includes an endplate preparation device guide. The endplate preparation device is adapted to modify the adjacent endplates of the first and second vertebrae for receiving a spinal stabilization implant and has a range of motion limited by the guide in at least one dimension.

In another embodiment, the invention includes a method for preparing adjacent endplates of first and second adjacent vertebrae for receiving a spinal stabilization implant. The method includes the steps of bilaterally engaging a first vertebra with spaced apart vertebra engaging portions of a first distraction arm of a surgical device, bilaterally engaging a second vertebra adjacent the first vertebra with spaced apart vertebra engaging portions of a second distraction arm of the surgical device, moving the first distraction arm with respect to the second distraction arm to increase a spacing between the adjacent endplates of the first and second adjacent vertebrae to at least a predetermined value, and guiding an endplate preparation device with a template portion of the surgical device while maintaining the spacing between adjacent endplates of the first and second vertebrae substantially at the predetermined value.

### BRIEF DESCRIPTION OF THE DRAWINGS

Other objects and advantages of the invention will become apparent upon reading the following detailed description and upon reference to the drawings in which:

FIG. 1 is a perspective view of a vertebral spreader and an endplate preparation template in the relative positions as they would be when the spreader and template are fully inserted into the disc space according to one aspect of the invention;

FIG. 2 is a perspective view of the vertebral spreader and endplate preparation template shown in FIG. 1 but from a different angle;

FIG. 3 is a more detailed view of tip portions of the vertebral spreader and the endplate preparation template shown in FIG. 1, additionally showing a reamer and associated sleeves;

FIG. 4 schematically shows a side view of an endplate preparation template inserted between two adjacent vertebrae, with a reamer inserted into the disc space; and

FIG. 5 is a perspective view of a vertebral spreader and an endplate preparation template inserted between two adjacent vertebrae.

### DETAILED DESCRIPTION OF SPECIFIC EMBODIMENTS

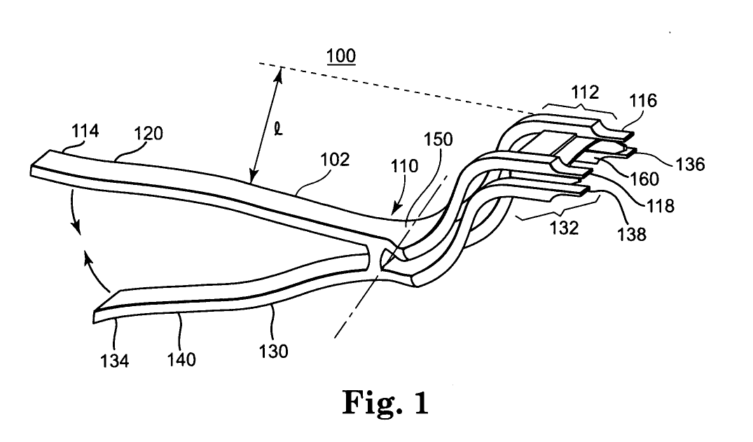

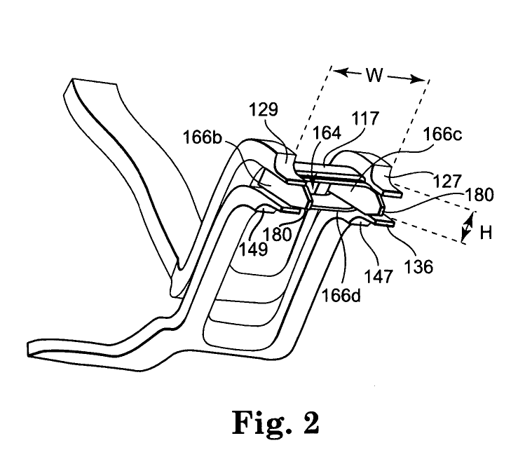

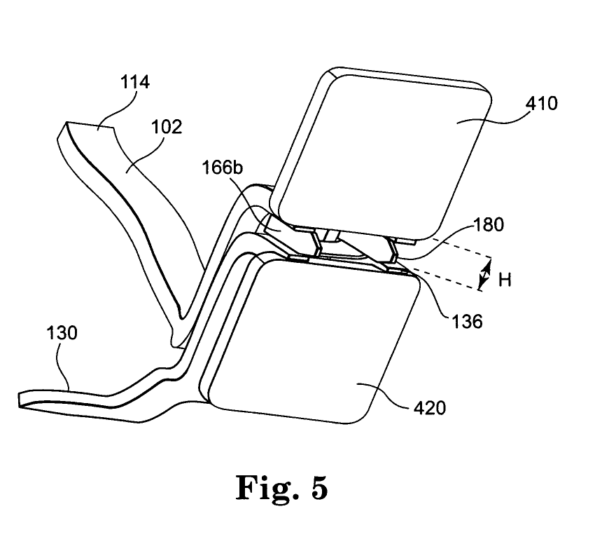

In one embodiment of the invention, illustrated in FIGS. 1-5, a surgical instrument system 100 may include a vertebral spreader 110 and a template 160. The spreader 110 is used to displace two adjacent vertebrae 410, 420 apart from each other, as illustrated in FIG. 5. The spreader 110 includes a first distraction arm 102 and a second distraction arm 130. The first distraction arm 102 has a distal end 112 and a proximal end 114. The distal end 112 may include a pair of vertebra engaging portions, such as prongs 116 and 118, for bilaterally engaging and supporting the upper vertebra 103 at two positions spaced apart laterally from each other. A crossbar 117 interconnects the prongs 116 and 118 for increased rigidity of the distal end 112. The proximal end 114 includes a handle portion 120. Similarly, the second distraction arm 130 has a distal end 132 and a proximal end 134. The distal end 132 may include a pair of prongs 136 and 138 for bilaterally engaging and supporting the lower vertebra 104. A crossbar (not shown) similar to the crossbar 117 interconnects the prongs 136 and 138 for increased rigidity of the distal end 132. The proximal end 134 includes a handle portion 140.

The first and second distraction arms 102, 130 are joined to each other in this embodiment by a pivotal connection 150 in such a way that when the handle portions 120, 140 are brought closer toward each other, the distal ends 112, 132 spread apart. The distance between the two vertebrae can therefore be increased by forcing the handle portions 120, 140 toward each other. Other types of connections can also be used, depending on the specific procedure desired.

In one embodiment, each of the prongs 116, 118, 136, 138 includes a recessed portion 126, 128, 146, 148, respectively, at the tip of prong. The recessed portions 126, 128, 146, 148 may include edges 127, 129, 147, 149 that are able to engage with the sides of the respective vertebrae. In this embodiment, when the recessed portions 126, 128, 146, 148 of the prongs 116, 118, 136, 138 are inserted a certain distance into a disc space, the edges 127, 129, 147, 149 engage with the sides of the respective vertebrae and stop the spreader 110 from moving further into the disc space.

The template 160 may also include a plurality of spacer portions that define a volume of space and are adapted to bilaterally engage both the first and second vertebrae and maintain the position of the vertebrae at a predetermined distance from each other. In one embodiment the spacer portions are formed of two paddles 180, 200. The template may also include a guide portion 162, or an endplate preparation device guide, from which the two paddles 180, 200 project. The guide portion 162 in one embodiment has an opening 164 enclosed by two side walls 166a, 166b, an upper wall 166c and a lower wall 166d, and has a height H and width W. The paddles 180, 200 extend from the side walls 166a, 166b, respectively, and may be inserted in between the upper and lower vertebrae. Paddle 180 has a proximal region 182 with a height h between the substantially parallel top and bottom edges 184, 186 for maintaining the distance between the top and bottom vertebrae at a distance h from each other. The height h in this case is smaller than the height H of the opening 164 of the guide portion 162 but can be other sizes relative to the opening 164 depending on the specific surgical needs. Paddle 180 may include a tapered distal region 188, with a gradually decreasing height toward the distal end 190 to facilitate insertion of the paddle 180 into the disc space between the vertebrae. Similarly, paddle 200 may have a proximal region 202 with a height h between the substantially parallel top and bottom edges 204, 206. Paddle 200 may also have a tapered distal portion 208, with a gradually decreasing height toward the distal end 210 to facilitate insertion of the paddle 200 into the disc space between the vertebrae. Other configurations of the paddles 180, 200 may be used. For example, the proximal regions 182, 202 or the entire paddles 180, 200 may be tapered to maintain a lordotic relationship between the top and bottom vertebrae when the template 160 is inserted from a posterior approach. The proximal portions 182, 202 can also have different heights to maintain a lordotic relationship between the top and bottom vertebrae when the template 160 is inserted from a lateral approach. A combination of different heights and degrees of tapering can be used to accomplish the desired anatomy and surgical approach.

As shown in FIGS. 1-3 and 5, the distal end 112 of the first distraction arm 102 may be offset from the proximal end 114 by a distance 1. The distal end 132 of the second distraction arm 130 may be similarly offset from the proximal end 134. The offset may be sufficiently large to permit access to the disc space by the template 160, the elongated endplate preparation devices (described below) and other instruments from the same side of, in one embodiment, the handle portions 120, 140. Such access facilitates ease of operation and optimum visibility of the surgical site. Other configurations of the vertebral spreader can be used to achieve the desired characteristics. For example, the handle portions 120, 140 need not be substantially parallel to the respective pairs of prongs 116, 118 and 136, 138, but can be instead at other angles, such as from about 90° to about 125° from the respective pairs of prongs.

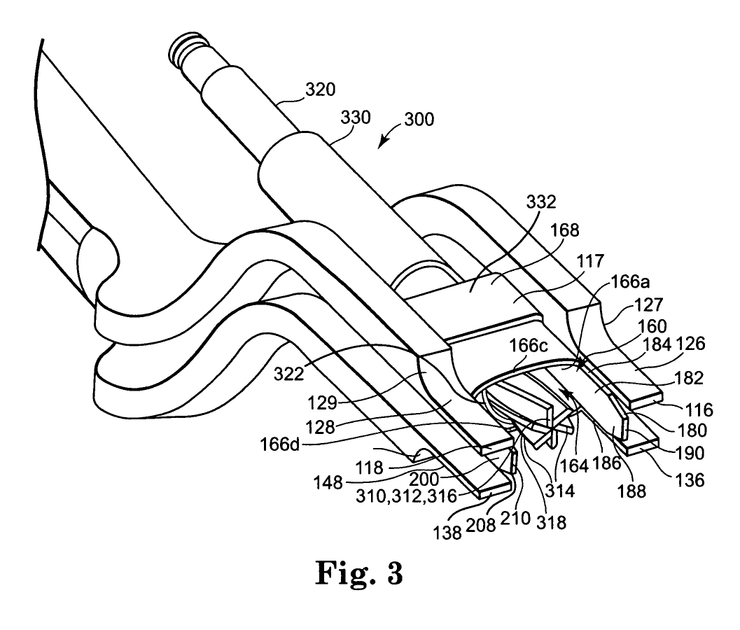

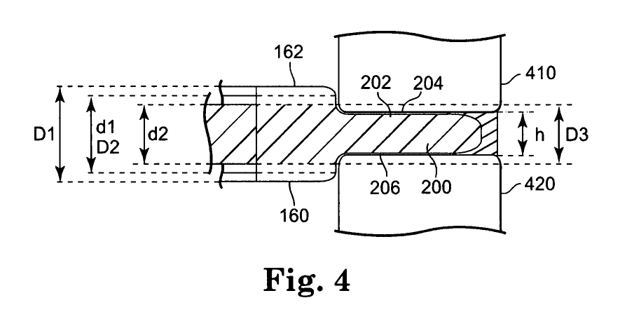

Referring more specifically to FIGS. 3 and 4, in one embodiment of the invention, the surgical instrument system 100 may also include an endplate preparation device 300, which in this case includes a cutter 310, an inner sleeve 320 and an outer sleeve 330. The outer sleeve 330 may have an outer diameter D1, which is larger than the height H of the opening 164 of the template 160 so that the distal end 332 of the outer sleeve 330 is prevented from advancing beyond the proximal end 168 of the guide portion 162 of the template 160 toward the disc space. The inner sleeve 320 may have an outer diameter D2, which may be smaller than, or approximately equal to, the inner diameter dl of the outer sleeve 330. Furthermore, the outer diameter D2 of the inner sleeve 320 may be smaller than, or approximately equal to, the height H of the opening 164 so that the distal end 322 of the inner sleeve 320 can be inserted through the opening 164 toward the disc space. The depth of advancement of the distal portion 322 of the inner sleeve 320 toward the disc space may be controlled in any suitable ways, including using the outer sleeve 330 as a guide or stop. For example, the inner and outer sleeves 320, 330 may be made to have predetermined lengths such that the end of the inner sleeve advancement toward the disc space is indicated by the coincidence between the proximal end (not shown) of the inner sleeve 320 and the proximal end (not shown) of the outer sleeve 330. The inner sleeve 320 may also include a flange or other types of stops at or near the proximal end to prevent the advancement of the inner sleeve 320 beyond a predetermined point relative to the outer sleeve 330. Scale marks may also be placed on either sleeve to indicate the relative position between the two sleeves.

The cutter 310 in one embodiment may include a reamer bit 312, with cutting edges 314 on both the distal end surface 316 and side surface 318. The reamer bit in this case may be held by the chuck of a power drive (not shown). The reamer bit 312 may have a diameter D3, which is smaller, or substantially equal to the inner diameter d2 of the inner sleeve 320. In one embodiment illustrated in FIG. 4, the diameter D3 of the reamer bit in this case is greater than the height h of the paddles 180, 200 so that the reamer bit 312 cuts into the vertebrae 410, 420, which are spaced apart by the paddles 180, 200. The advancement of the reamer bit 312 may be controlled by using a variety of indicators or stops as described above for the inner sleeve 320. The chuck holding the reamer bit 312 may also act as a stop limiting the forward travel of the reamer bit 312.

In one embodiment, as illustrated in FIGS. 4 and 5, the paddles 180, 200 may be inserted between the upper and lower vertebrae 410, 420 after the vertebrae 410, 420 are pushed sufficiently far apart by the vertebral spreader 110. The paddles 180, 200 are able to maintain the vertebrae 410, 420 at distance h apart. The reamer bit 312, inner sleeve 320 and outer sleeve 330 are then introduced to the disc space. The outer sleeve 330 may then be advanced up to the proximal end 168 of the template 160. The inner sleeve 320 may be placed inside the outer sleeve 330 and advanced into the guide portion 162 of the template 160. The reamer bit 312 may then be placed inside the inner sleeve 320 and advanced into the disc space for removing material from the vertebrae 410, 420 while being shielded elsewhere by the sleeves 320, 330. The reamer bit 312 and the inner sleeve 320 may be allowed to move in at least one direction transverse to the longitudinal axis of the reamer bit 312 but the transverse movement may be limited by the walls 166a, 166b, 166c, 166d of the guide portion 162. The endplates of the vertebra 410, 420 may thus be quickly and accurately modified by the reamer bit 312. The intended implant or implants may then be inserted into the disc space.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. A surgical instrument system for distraction and endplate preparation of adjacent vertebrae during a spinal stabilization procedure, the surgical instrument system comprising:
a first distraction arm comprising a first pair of spaced apart vertebra engaging portions at a distal end of the first arm wherein each vertebra engaging portion is adapted to bilaterally engage a first vertebra;
a second distraction arm comprising a second pair of spaced apart vertebra engaging portions at a distal end of the second arm wherein each vertebra engaging portion is adapted to bilaterally engage a second vertebra adjacent the first vertebra, the distal ends of the first and second arms being movable with respect to one another; and
a template sized to be inserted between each pair of the spaced apart vertebra engaging portions, wherein the template is adapted to bilaterally engage both the first and second vertebrae and maintain the position of the vertebrae at a predetermined distance from each other.

2. The surgical instrument system of claim 1, wherein the template comprises a pair of spacer portions spaced apart from each other, defining a volume of space between them, and positionable between the two vertebrae, the spacer portions being adapted to bilaterally engage both the first and second vertebrae and maintain the position of the vertebrae apart at a predetermined distance from each other.

3. The surgical instrument system of claim 2, wherein the template further comprises a guide portion, and the pair of spacer portions are attached to the guide portion, the guide portion defining an opening through which the volume of space between the pair of spacer portions can be accessed.

4. The surgical instrument system of claim 3, further comprising an elongated cutter positionable through the opening of the guide portion of the template into the volume of space between the pair of spacer portions and adapted to remove material from the vertebrae.

5. The surgical instrument system of claim 4, wherein the guide portion comprises walls adapted to limit lateral movement of the cutter when removing material from the vertebrae.

6. The surgical instrument system of claim 5, further comprising a sleeve sized to slip over the cutter and positionable inside the opening.

7. The surgical instrument system of claim 6, wherein the sleeve comprises a stop configured to engage the template to prevent the sleeve from advancing beyond a predetermined position toward the vertebrae.

8. The surgical instrument system of claim 1, wherein each distraction arm further comprises an elongated handle at a proximal end, the distal ends of both distraction arms being offset from the handles along a direction generally transverse to the handles.

9. The surgical instrument system of claim 8, wherein the distraction arms are pivotally connected to each other.

10. The surgical instrument system of claim 9, wherein the distraction arms are configured to move their distal ends away from each other when the handles move toward each other.

11. A surgical system for distraction and endplate preparation of adjacent vertebrae during a spinal stabilization procedure, the surgical system comprising:
a distraction device, the distraction device comprising:
a first distraction arm comprising first and second spaced apart vertebra engaging portions at a distal end, wherein the first and second spaced apart vertebra engaging portions are configured for bilaterally engaging a first vertebra;
a second distraction arm comprising first and second spaced apart vertebra engaging portions at a distal end, wherein the first and second spaced apart vertebra engaging portions of the second distraction arm are configured for bilaterally engaging a second vertebra that is adjacent the first vertebra, and wherein the second distraction arm is movable with respect to the first distraction arm for separating adjacent endplates of the first and second vertebrae; and
an endplate preparation device guide; and
an endplate preparation device adapted to modify the adjacent endplates of the first and second vertebrae for receiving a spinal stabilization implant, the endplate preparation device having a range of motion limited by the guide in at least one dimension.

12. The surgical system of claim 11, further comprising a plurality of spacers attached to the endplate preparation device guide, the spacers being positionable between the adjacent vertebrae for maintaining a predetermined spacing between the vertebrae.

13. The surgical system of claim 12, wherein the plurality of spacers are spaced apart from each other, defining a space for accommodating at least a portion of the endplate preparation device.

14. The surgical system of claim 11, wherein the endplate preparation device comprises a reamer bit.

15. A method for preparing adjacent endplates of first and second adjacent vertebrae for receiving a spinal stabilization implant, the method comprising the steps of:
bilaterally engaging a first vertebra with spaced apart vertebra engaging portions of a first distraction arm of a surgical device;
bilaterally engaging a second vertebra adjacent the first vertebra with spaced apart vertebra engaging portions of a second distraction arm of the surgical device;
moving the first distraction arm with respect to the second distraction arm to increase a spacing between the adjacent endplates of the first and second adjacent vertebrae to at least a predetermined value; and
guiding an endplate preparation device with a template portion of the surgical device while maintaining the spacing between adjacent endplates of the first and second vertebrae substantially at the predetermined value.

16. The method of claim 15, further comprising modifying at least one of the adjacent endplates of the first and second vertebrae with the endplate preparation device.

17. The method of claim 16, wherein the modifying step further comprises passing the endplate preparation device through an opening in the template portion into a position between the two spacer portions.

18. The method of claim 17, wherein the step of passing the endplate preparation device through an opening in the template portion further comprises passing the endplate preparation device through the opening on the same side of both of the distraction arms.

19. The method of claim 15, wherein maintaining the spacing between adjacent endplates of the first and second vertebrae at the predetermined value comprises disposing two spaced-apart spacer portions of the template between the adjacent endplates.

20. The method of claim 15, wherein the guiding step further comprises using the template portion to stop the endplate preparation device from advancing beyond a predetermined point relative to the template.
